(19) United States
(12) Patent Application Publication
(10) Pub. No.: US 2026/0000909 A1
(43) Pub. Date: Jan. 1, 2026

(54) Light-Based Hair Growth Apparatus

(71) Applicant: Ever Wynn Technology Co., Ltd., Taichung (TW)

(72) Inventor: Yu-Cheng Lee, Taichung (TW)

(21) Appl. No.: 19/023,240

(22) Filed: Jan. 15, 2025

(30) Foreign Application Priority Data
Jul. 1, 2024 (TW) 113207050
Dec. 4, 2024 (TW) 113213325

(51) Int. Cl. A61N 5/06; A61N 5/067

(52) U.S. Cl. CPC A61N 5/0617; A61N 5/067; A61N 2005/0627; A61N 2005/0632; A61N 2005/0647; A61N 2005/0651; A61N 2005/0659; A61N 2005/0663

(57) ABSTRACT

A light-based hair growth apparatus includes a cap body having an inner surface, a flexible circuit board disposed on the inner surface of the cap body, the first light-emitting component and the second light-emitting component disposed on the flexible circuit board, and a controller electrically coupled to the flexible circuit board. The controller used to control alternative irradiation of the first and the second light-emitting components and to control the flash irradiation duration and the number of flashes per second of the individual light-emitting components.

### TECHNICAL FIELD

The present invention relates to a hair therapy, and more particularly, a light-based hair growth apparatus.

### BACKGROUND

Hair diseases, including baldness and hair loss (alopecia), occur commonly in adults. With the advancement of medical technology, the methods for treating baldness and hair loss are becoming increasingly diversified. Clinically, they can be roughly divided into two categories, i.e., hair growth treatment and hair transplant surgery. Among them, in addition to hair growth toner and oral medication treatments, the hair growth treatment also includes various physical therapies and laser hair growth treatments, which can effectively slow down the baldness and hair loss.

Laser light irradiation has been used in medicine treatments for many years. The wavelength of laser light used in medicine is ranged between 600 nm to 1000 nm. It has strong penetrating power and radiation energy, having significant temperature control and resonance effects, which can be easily absorbed by objects and converted into internal energy of matter.

Sebaceous glades below hair follicles can be shrunk by utilizing a low-power laser to stimulate and irradiate the hair follicles, therefore reducing oil secretion, and hair loss caused by excessive oil secretion can be improved. At the same time, the low-power laser irradiation being introduced to the hair follicles can accelerate hair growth and enhance the firmness, density and elasticity of the hair itself.

Currently, majority of the laser-based hair growth devices adopt a wear-type design. In these devices, multiple light-emitting modules can be installed on a cap body. The multiple light-emitting modules can emit laser beams to illuminate the user's head for stimulating the user's hair follicles, thereby stimulating hair regrowth.

Most of the existing laser-based hair growth devices apply laser diodes to irradiate the user's scalp, but have only one simple functionality showing slow hair regrowth and poor therapeutic effects.

Therefore, a novel hair growth device is needed in view of the shortcomings of the existing technology.

### SUMMARY OF THE INVENTION

In one aspect of the present invention, a light-based hair growth apparatus includes a cap body having an inner surface, a flexible circuit board, including the first light-emitting component and the second light-emitting component, being disposed on the inner surface of the cap body, and a controller electrically coupled to the flexible circuit board. The controller is configured to control alternative irradiations, the flash irradiation duration and the flashing frequency of the first light-emitting component and the second light-emitting component.

In one preferred embodiment, the first light-emitting component includes a plurality of laser generating units, and the second light-emitting component includes a plurality of infrared light generating units. The plurality of laser generating units and the plurality of infrared light generating units are interleaved and evenly disposed on the flexible circuit board. The plurality of laser generating units include red light laser diodes. The plurality infrared light-generating units includes infrared light-emitting diodes. Each of the red light laser diodes is configured to generate red light irradiation with a wavelength in a range of 630-680 nm. Each of the infrared light-emitting diodes is configured to generate infrared light irradiation with a wavelength in a range of 830-900 nm. The light-based hair growth apparatus further includes a power source configured to provide power to the first light-emitting component and the second light-emitting component.

In one preferred embodiment, the first light-emitting component and the second light-emitting component are configured to generate light irradiance in an interactive flashing manner. The first light-emitting component and the second light-emitting component are configured to flash one time in every five seconds with an irradiation cycle of 15 to 45 minutes.

In one preferred embodiment, the flexible circuit board includes a plurality of cuts, enabling the flexible circuit board to form a plurality of petal-like shapes while being flattened out. The cuts include at least two to eight cuts. Each cut includes curved cut, angled cut, non-curved cut, non-angled cut.

In one preferred embodiment, the light-based hair growth further includes a sensor disposed on the inner surface of the cap body, being configured to activate the first light-emitting component and the second light-emitting component when a user's scalp to be treated is placed within a predetermined distance.

In one preferred embodiment, the light-based hair growth apparatus further includes a soft light-transmitting layer disposed on the inner surface of the cap body to cover the flexible circuit board as a pad.

### BRIEF DESCRIPTION OF THE DRAWINGS

The components, characteristics and advantages of the present invention may be understood by the detailed descriptions of the preferred embodiments outlined in the specification and the drawings attached:

FIG. 1 shows the appearance of the light-based hair growth apparatus disclosed by the present invention.

FIG. 2 shows a diagram of an internal view of the light-based hair growth apparatus according to one embodiment of the present invention.

FIG. 3 shows a cross-sectional diagram of the light-based hair growth apparatus according to one embodiment of the present invention.

FIG. 4 shows a configuration diagram of the first light-emitting component and the second light-emitting component disposed on the flexible circuit board according to one embodiment of the present invention.

FIG. 5 shows a functional block diagram of the light-based hair growth apparatus according to one embodiment of the present invention.

### DETAILED DESCRIPTION

Some preferred embodiments of the present invention will now be described in greater detail. However, it should be recognized that the preferred embodiments of the present invention are provided for illustration rather than limiting the present invention. In addition, the present invention can be practiced in a wide range of other embodiments besides those explicitly described, and the scope of the present invention is not expressly limited except as specified in the accompanying claims.

Alopecia, or hair loss, which is a common condition that affects 50% of men over 40 years old and 75% of women over 65 years old, has few proven effective treatments. The present invention provides red light low-level laser therapy (LLLT) to stimulate user's hair growth. In one preferred embodiment, the present invention applies red light with wavelength in a range of 630-680 nm to stimulate epidermal stem cells of hair follicles of the user and transform the hair follicles to the anagen phase (the phase of active hair growth). The present invention applies light with wavelength in a range of 830-900 nm, which is in the wavelength range of infrared light, to improve the user's blood circulation (blood flow) and promote the absorption of essence applied to the user's scalp.

The present invention applies red light low-level laser therapy (LLLT) to the user for increasing the user's blood vessel diameter and improving the user's blood circulation. In addition, the red light low-level laser therapy (LLLT) can also be used to protect the user's red blood cells from oxidative stress and reduce the user's platelet loss during surgical procedures.

The present discloses a light-based hair growth apparatus, which mainly combines red light (wavelength range: 630-680 nm) low-level laser therapy (LLLT) and infrared light (wavelength range: 830-900 nm) therapy, used to respectively stimulate scalp hair follicles and blood circulation, thereby promoting healthy hair growth. By transmitting safe and effective light energy to the scalp, the scalp is exposed to the low-energy laser light irradiation for promoting regeneration to the weakened hair follicles.

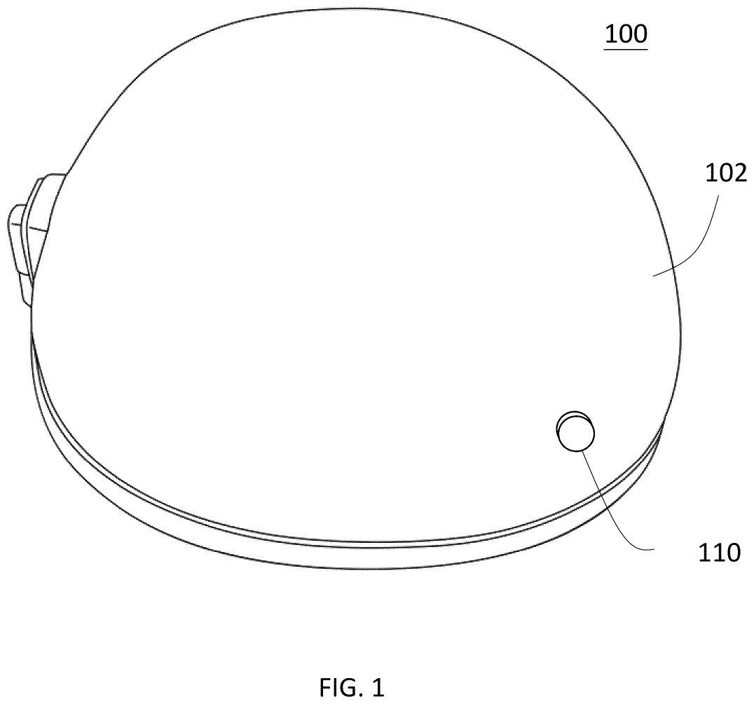

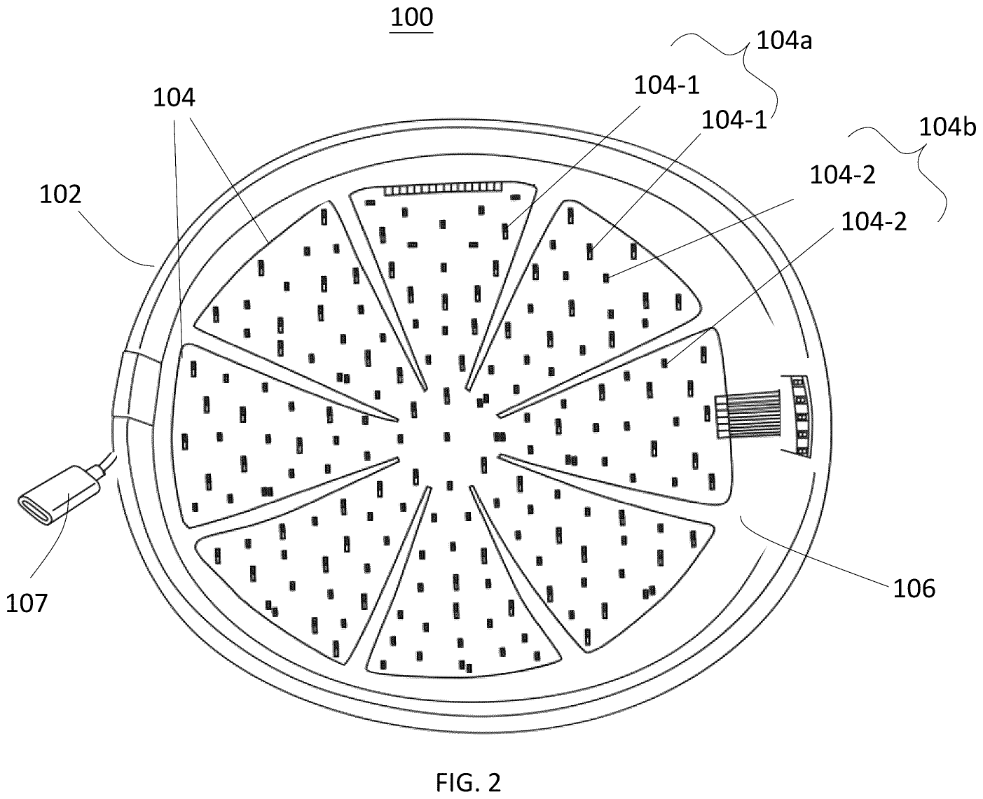

Referring to FIGS. 1-2, the light-based hair growth apparatus 100 of the present invention includes a cap body 102, such as a helmet, to act as the main body of the device, a flexible circuit board 104 disposed on the inner surface of the helmet, and a soft light-transmitting layer 106 disposed on the inner surface of the helmet to cover the flexible circuit board 104 and to serve as a pad. The flexible circuit board 104 is disposed with the first light-emitting component 104a and the second light-emitting component 104b, which are configured to alternately generate light irradiations with two different wavelengths. The cap body 102 includes an inner surface, an outer surface and an opening. The inner surface and the outer surface are respectively located on opposite sides of the cap body 102. A start button 110 is disposed on the outside thereof for starting the light-based hair growth apparatus 100. The flexible circuit board 104 is connected to an external power supply through a Type C power socket 107. In another embodiment, a battery may be applied to provide power, and the battery may be a dry cell or a rechargeable battery.

According to an embodiment of the present invention, the first light-emitting component 104a includes a plurality of laser light generating units 104-1, such as laser diodes, for generating red light irradiation with a wavelength in a range of 630-680 nm; the second light-emitting component 104b includes a plurality of infrared light generating units 104-2, such as light-emitting diodes (LEDs), for generating infrared light irradiation with a wavelength in a range of 830-900 nm. The plurality of laser light generating units 104-1 and the plurality of infrared light generating units 104-2 are interleaved and evenly disposed on the flexible circuit board 104.

According to an embodiment of the present invention, the laser diode delivers red light irradiance with wavelength in a range of 630-680 nm on the user's scalp. Each laser diode provides a beam of coherent radiation, while the light-emitting diodes (LEDs) provide more general and uniform light irradiation to stimulate the user's scalp.

According to an embodiment of the present invention, the first light-emitting component (630-680 nm laser diodes) 104a and the second light-emitting component (830-900 nm LEDs) 104b are configured to generate light irradiance in an interactive flashing manner. In some embodiments, the first light-emitting component 104a and the second light-emitting component 104b are configured to flash one time in every five seconds with an irradiation cycle of 15 to 45 minutes. In an embodiment, the first light-emitting component 104a and the second light-emitting component 104b are respectively light up for 12.5*60=750 seconds in one irradiation cycle.

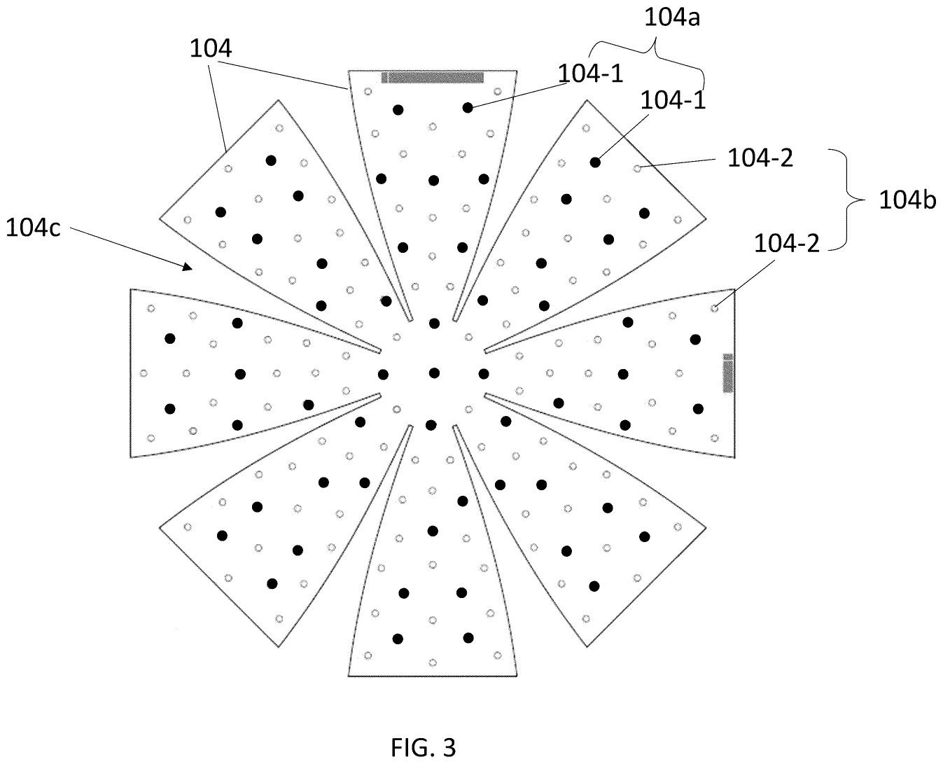

Please refer to FIG. 3, it shows a schematic cross-sectional diagram of the light-based hair growth apparatus 100 disclosed by this invention. The diagram shows, from outside to inside (top to bottom), the cap body (helmet) 102, the flexible circuit board 104 and the soft light-transmitting layer 106. The flexible circuit board 104 includes the first light-emitting component (630-680 nm laser diodes) 104a and the second light-emitting component (830-900 nm LEDs) 104b.

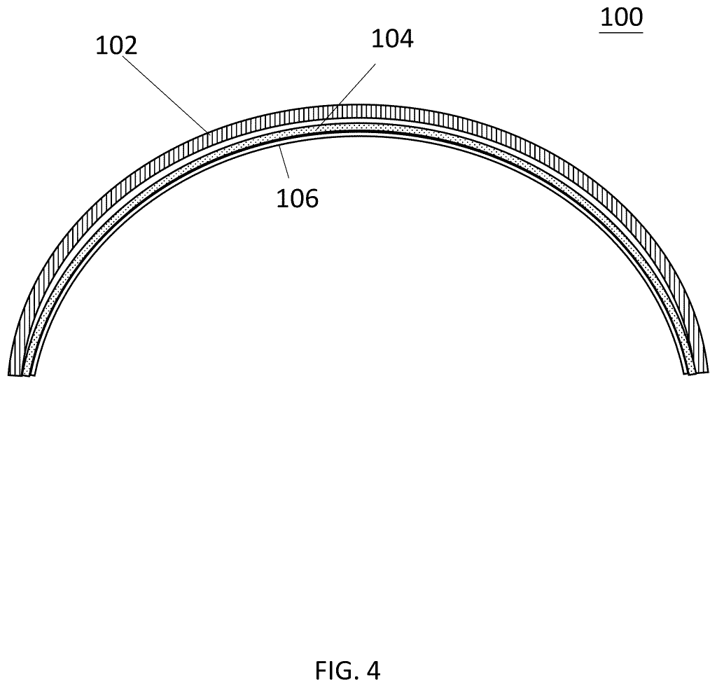

FIG. 4 shows a configuration diagram of the first light-emitting component 104a and the second light-emitting component 104b according to an embodiment of the present invention. In FIG. 4, the first light-emitting component 104a and the second light-emitting component 104b are distributed on the flexible circuit board 104, where the flexible circuit board 104 can be bent into a dome shape. The flexible circuit board, being disposed with the first light-emitting component 104a and the second light-emitting component 104b, is assembled inside the cap body 102 and conforms to the contour of the user's head, enabling the light-based hair growth apparatus 100 accurately target the affected area for treatment. In an embodiment, the flexible circuit board 104 may include a plurality of cuts 104c that are spaced apart to facilitate forming the flexible circuit board 104 into a dome shape that conforms to the contours of the user's head. In some embodiments, the plurality of cuts may include at least two cuts, such as at least three cuts, or at least four cuts, or at least five cuts, or at least six cuts, or at least eight cuts, enabling the flexible circuit board 104 to form a plurality of petal-like shapes while being flattened out. In some embodiments, the plurality of cuts may include curved cuts, angled cuts, non-curved cuts, non-angled cuts, and/or their combinations.

As shown in FIG. 4, the flexible circuit board 104 is disposed with a plurality of laser light generating units 104-1, such as laser diodes, configured to generate red light irradiation with a wavelength in the range of 630-680 nm; and a plurality of infrared light generating units 104-2, such as light emitting diodes (LEDs), configured to generate infrared light irradiation with a wavelength in the range of 830-900 nm. The plurality of laser light generating units 104-1 and the plurality of infrared light generating units 104-2 are interleaved and evenly disposed on the flexible circuit board 104.

FIG. 4 shows an embodiment of a flexible circuit board 104, which is connected to 176 light sources, preferably 58 laser diodes and 118 light-emitting diodes. In the embodiment, the light sources are evenly distributed on the flexible circuit board 104.

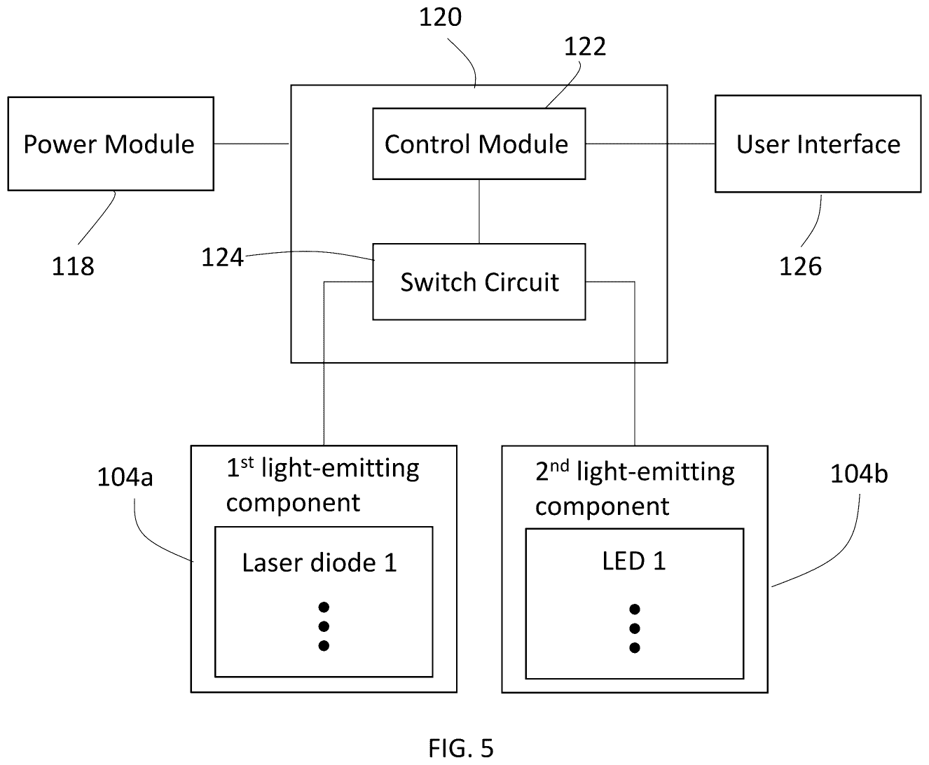

FIG. 5 shows a functional block diagram of the light-based hair growth apparatus 100 including a controller 120 according to one embodiment of the present invention. The power module 118 is electrically coupled to the controller 120 for providing power to the first light-emitting component 104a and the second light-emitting component 104b of the light-based hair growth apparatus 100. The controller 120 includes a control module 122 and a switch circuit 124. Each laser diode of the first light-emitting component 104a and each light-emitting diode of the second light-emitting component 104b may be directly electrically coupled to the switch circuit 124, and the switch circuit 124 is electrically coupled to the control module 122. The control module 122 may be implemented as a processor, an ASIC, a hardware controller, or the like, which may be programmed or otherwise configured to provide control signals. Therefore, the control module 122 may provide software instructions to implement certain functions, such as the function of flash irradiation.

The controller 120 controls the operation of the light-based hair growth apparatus 100. The controller 120 provides control signals to the switch circuit 124, through the control module 122, for activating or deactivating the first light-emitting component 104a and the second light-emitting component 104b, according to the desired operating sequence or mode in combination with a timer. The desired operating sequence or mode may include, such as the flashing irradiation sequence of the first light-emitting component 104a and the second light-emitting component 104b, the flashing irradiation duration of the individual light-emitting components, and the flashing frequency (flashes per second). The user interface 126 may be coupled to the control module 122. The user interface 126 allows the user to select an operating mode, or in some embodiments allows the user to set the operation of the light-based hair growth apparatus 100. In other embodiments, other wiring or switches may be used to produce different startup modes.

While various embodiments of the present invention have been described above, it should be understood that they have been presented by a way of example and not limitation. Numerous modifications and variations within the scope of the invention are possible. The present invention should only be defined in accordance with the following claims.

What is claimed is:

1. A light-based hair growth apparatus, comprising:
a cap body having an inner surface;
a flexible circuit board disposed on said inner surface of said cap body, said flexible circuit board including a first light-emitting component and a second light-emitting component; and
a controller electrically coupled to said flexible circuit board to control a flash irradiation duration and a flashing frequency of said first light-emitting component and said second light-emitting component.

2. The apparatus of claim 1, wherein said first light-emitting component includes a plurality of laser generating units; wherein said second light-emitting component includes a plurality of infrared light generating units.

3. The apparatus of claim 2, wherein said plurality of laser generating units and said plurality of infrared light generating units are interleaved and evenly disposed on said flexible circuit board.

4. The apparatus of claim 3, wherein said plurality of laser generating units include red light laser diodes; wherein said plurality infrared light generating units include infrared light-emitting diodes.

5. The apparatus of claim 4, wherein each of said red light laser diodes is configured to generate red light irradiation with a wavelength in a range of 630-680 nm; wherein each of said infrared light-emitting diodes is configured to generate infrared light irradiation with a wavelength in a range of 830-900 nm.

6. The apparatus of claim 5, further including a power source configured to provide power to said first light-emitting component and said second light-emitting component.

7. The claim 6, wherein said first light-emitting component and said second light-emitting component are configured to generate light irradiance in an interactive flashing manner.

8. The apparatus of claim 7, wherein said first light-emitting component and said second light-emitting component are configured to flash one time in every five seconds with an irradiation cycle of 15 to 45 minutes.

9. The apparatus of claim 1, wherein said flexible circuit board includes a plurality of cuts, enabling said flexible circuit board to form a plurality of petal-like shapes while being flattened out.

10. The apparatus of claim 9, wherein said plurality of cuts include at least two to eight cuts.

11. The apparatus of claim 10, wherein said plurality of cuts include curved cuts, angled cuts, non-curved cuts, non-angled cuts, and/or their combinations.

12. The apparatus of claim 1, further including a sensor disposed on said inner surface of said cap body to activate said first light-emitting component and the second light-emitting component when said sensor detects a user's scalp within a predetermined distance.

13. The apparatus of claim 1, further including a soft light-transmitting layer disposed on said inner surface of said cap body to cover said flexible circuit board as a pad.

14. A light-based hair growth apparatus, comprising:
a cap body having an inner surface;
a flexible circuit board disposed on said inner surface of said cap body, said flexible circuit board including a first light-emitting component and a second light-emitting component; wherein said first light-emitting component includes a plurality of laser generating units, said second light-emitting component including a plurality of infrared light generating units; and
a controller electrically coupled to said flexible circuit board to control a flash irradiation duration and a flashing frequency of said first light-emitting component and said second light-emitting component;
wherein said plurality of laser generating units and said plurality of infrared light generating units are interleaved and evenly disposed on said flexible circuit board.

15. The apparatus of claim 14, wherein said plurality of laser generating units include red light laser diodes; wherein said plurality infrared light generating units include infrared light-emitting diodes.

16. The apparatus of claim 15, wherein each of said red light laser diodes is configured to generate red light irradiation with a wavelength in a range of 630-680 nm; wherein each of said infrared light-emitting diodes is configured to generate infrared light irradiation with a wavelength in a range of 830-900 nm.

17. The apparatus of claim 16, further including a power source configured to provide power to said first light-emitting component and said second light-emitting component.

18. The claim 17, wherein said first light-emitting component and said second light-emitting component are configured to generate light irradiance in an interactive flashing manner.

19. The apparatus of claim 18, wherein said first light-emitting component and said second light-emitting component are configured to flash one time in every five seconds with an irradiation cycle of 15 to 45 minutes.

20. The apparatus of claim 14, wherein said flexible circuit board includes a plurality of cuts, enabling said flexible circuit board to form a plurality of petal-like shapes while being flattened out.
